(19) United States
(12) Patent Application Publication
(10) Pub. No.: US 2003/0149038 A1
(43) Pub. Date: Aug. 7, 2003

(54) Alpha-helix mimicry by a class of organic molecules

(75) Inventors: Rodney K. Guy, San Francisco, CA (US); Irwin D. Kuntz, Greenbrae, CA (US); Felice Lu, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/293,179

(22) Filed: Nov. 12, 2002

Related U.S. Application Data
(60) Provisional application No. 60/339,239, filed on Nov. 9, 2001.

(51) Int. Cl. A61K 31/5377; A61K 31/519; A61K 31/53; A61K 31/52; A61K 31/502; A61K 31/498; A61K 31/4709; A61K 31/444; C07D 413/14; C 07D 4 3/14

(52) U.S. Cl. 514/234.5; 514/242; 514/252.02; 514/248; 514/249; 514/263.22; 514/314; 514/307; 514/332; 514/333; 544/112; 544/115; 544/117; 544/238; 544/236; 544/277; 544/350; 544/353; 544/405; 546/148; 546/167; 546/256

(57) ABSTRACT

The present invention provides methods for making compounds and methods for using the compounds to disrupt or inhibit protein-protein interactions. Also provided are pharmaceutical compositions comprising the compounds of the current invention.

### CROSS-REFERENCES TO RELATED APPLICATIONS

This application claims priority to U.S. S No. 60/339,239 filed Nov. 9, 2001, which is incorporated herein by reference.

### STATEMENT AS TO INVENTION MADE UNDER FEDERALLY SPONSORED RESEARCH AND DEVELOPMENT

This application is partially funded by the government of the Unites States of America (NIH), Contract No. GM 31497.

### FIELD OF THE INVENTION

This invention pertains to the field of inhibition or disruption of protein-protein interactions. Compounds that inhibit or disrupt protein-protein interactions as well as methods of making them are provided. Also provided are methods of inhibiting or disrupting protein-protein interactions as well as pharmaceutical compositions.

### BACKGROUND OF THE INVENTION

Progress in the treatment of solid tumors has been slow and sporadic despite the development of new chemotherapeutic agents. There are many roadblocks to successful chemotherapy, including drug resistance, resistance to apoptosis, and the inactivation of tumor suppressor genes. Some human cancers are drug resistant before treatment begins, while in others drug resistance develops over successive rounds of chemotherapy.

One type of drug resistance, called multidrug resistance, is characterized by cross resistance to functionally and structurally unrelated drugs. Typical drugs that are affected by the multidrug resistance are doxorubicin, vincristine, vinblastine, colchicine, actinomycin D, and others. At least some multidrug resistance is a complex phenotype that is linked to a high expression of a cell membrane drug efflux transporter called Mdrl protein, also known as P-glycoprotein. This membrane “pump” has broad specificity and acts to remove from the cell a wide variety of chemically unrelated toxins.

Another factor in cancer therapy is the susceptibility of targeted cells to apoptosis. Many cytotoxic drugs that kill cells by crippling cellular metabolism at high concentration can trigger apoptosis in susceptible cells at much lower concentration. Increased susceptibility to apoptosis can be acquired by tumor cells as a byproduct of the genetic changes responsible for malignant transformation, but most tumors tend to acquire other genetic lesions which abrogate this increased sensitivity. Either at presentation or after therapeutic attempts, the tumor cells can become less sensitive to apoptosis than vital normal dividing cells. Such tumors are generally not curable by conventional chemotherapeutic approaches. Although decreased drug uptake, altered intracellular drug localization, accelerated detoxification and alteration of drug target are important factors, pleiotropic resistance due to defective apoptotic response is also a significant category of drug resistance in cancer.

An important tumor suppressor gene is the gene encoding the cellular protein, p53, which is a 53 kD nuclear phosphoprotein that controls cell proliferation. Mutations to the p53 gene and allele loss on chromosome 17p, where this gene is located, are among the most frequent alterations identified in human malignancies. The p53 protein is highly conserved through evolution and is expressed in most normal tissues. Wild-type p53 has been shown to be involved in control of the cell cycle, transcriptional regulation, DNA replication, and induction of apoptosis.

Various mutant p53 alleles are known in which a single base substitution results in the synthesis of proteins that have quite different growth regulatory properties and, ultimately, lead to malignancies. In fact, the p53 gene has been found to be the most frequently mutated gene in common human cancers, and is particularly associated with those cancers linked to cigarette smoke. The overexpression of p53 in breast tumors has also been documented.

MDM2 binds to an alpha helix in the amino terminus of p53 and can prevent p53 from transcriptional signaling by either blocking function of the p53 transactivation domain or by targeting p53 for proteolytic degradation. Both inactivation of the p53 protein and over-expression of the MDM2 protein have been associated with increased tumor incidence in human patients (May et al., Oncogene 18: 7621-7236 (1999)). In particular, MDM2 is overexpressed in 20% of soft tissue tumors, 16% of osteosarcomas, 13% of esophageal carcinomas, and 8% astrocytomas (Momand et al., Nucleic Acids Res 26: 3453-3459 (1998)). Inhibitors of the MDM2-p53 interaction can be used to understand the role of p53 and MDM2 in cellular signaling.

p53 is involved in a regulatory feedback loop as well as a complex signaling pathway ending in cell cycle arrest and apoptosis (Stewart et al., Chem Res Toxicol 14: 243-263 (2001)). The regulatory feedback loop mainly involves p53's activation of MDM2, MDM2's suppression of p53, and p19^{ARF}'s suppression of MDM2. The signaling pathway downstream of p53 involves many gene products, most of which remain unidentified. Chemical inhibitors of the MDM2-p53 interaction would increase p53 levels, thereby activating downstream genes, which can be detected by techniques such as microarray analysis. Furthermore, the effect of p53 levels on upstream genes due to an undiscovered regulation loop could also be detected. p53 and MDM2's central role in signaling cell proliferation or death makes MDM2 inhibition studies valuable. To date, the discovery of chemical inhibitors of MDM2 has been limited to peptides containing key p53 residues (Bottger et al., Oncogene 13: 2141-2147 (1996)), piperazine-4-phenyl derivatives (Luke et al., Great Britain Patent No. WO00115657 (2000)), chalcones (Stoll et al., Biochemistry 40: 336-344 (2001)) and chlorofusin (Duncan et al., Journal of the American Chemical Society 123: 554-560 (2001)). Other than the peptides, none of these functions with an inhibitory constant stronger than 100 μM.

Despite the significance of p53 as a regulator of cellular growth/death and its apparent role in human disease, the details regarding its biological actions remain relatively obscure. Identifying new isoforms of p53 and defining how they affect cellular activity may lead to new ways of regulating cell growth and, eventually, to new diagnostic and therapeutic procedures. Thus, there is a need in the art for effective inhibitors or disruptors of the p53-MDM2 interaction. The present invention relieves this need by providing compounds that are of use as probes for investigating the function of p53 and for treating diseases associated with this protein.

### BRIEF SUMMARY OF THE INVENTION

The tumor suppressor p53 is a key protein involved in cellular response to DNA damage and oxidative stress. In response to stress, p53 activates many genes whose products lead to apoptosis or cell cycle arrest. The MDM2 protein regulates p53 transactivation by directly occluding p53's interaction with DNA and targeting p53 for degradation. In some cancers, overexpression of MDM2 leads to abnormal inactivity of p53, which promotes transformation. Molecules that disrupt or inhibit the binding of p53 to MDM2 are biological probes for investigating signaling events leading to apoptosis and cell cycle arrest and are useful as cancer therapies.

In a first aspect, the invention provides a compound having a formula selected from:

A-L-B-L¹-A¹

and

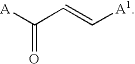

Substituent A is typically selected from the group:

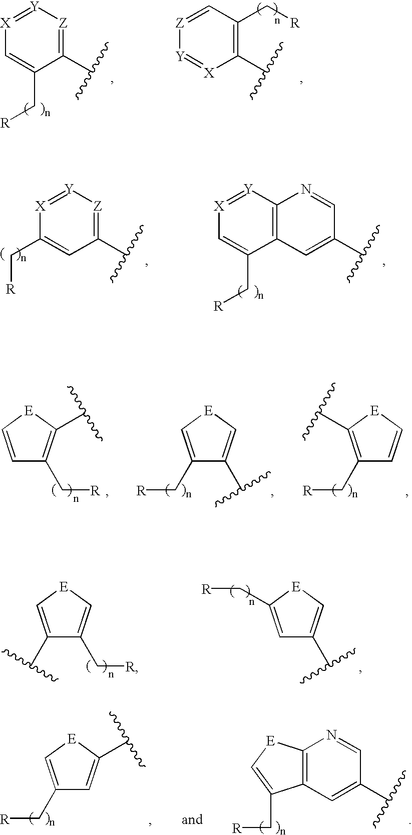

Substituent A¹ is typically selected from the group:

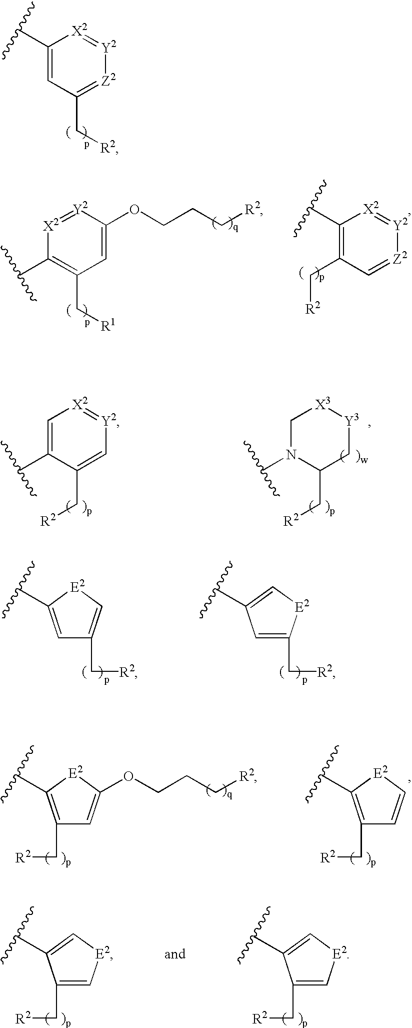

The core moiety, B, is typically selected from the group:

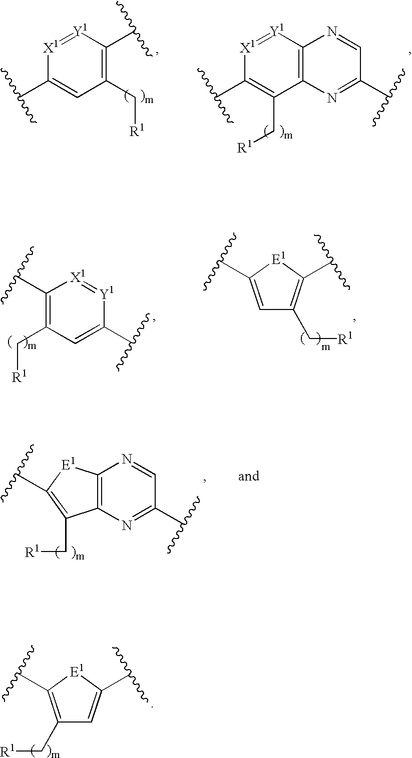

The linker moieties L and L¹ are typically selected from the group:

—N═N—, —CH₂—CH₂—, —C═C—, —CH₂—CH₂—, —CH₂—S—, —CH₂—NH—, —NH—CH₂,

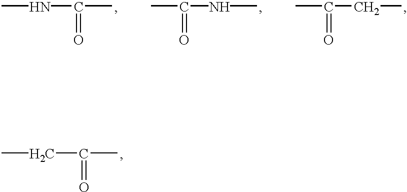

and a single bond.

The side group substituents R, R¹, and R² are typically selected from hydrogen, substituted or unsubstituted alkyl, substituted or unsubstituted heteroalkyl, substituted or unsubstituted aryl, substituted or unsubstituted neteroaryl, and substituted or unsubstituted heterocycloalkyl.

The ring moieties X, X¹, X², Y, Y¹, Y², Z, Z¹, and Z² are typically selected from —N— and —CH—. The ring moieties X³, Y³, E, E¹, and E² are typically selected from —NH—, —CH₂—, —S—, and —O—.

The parenthetical subscripts n, m, p and q are integers typically in the range from 0 to 4. Finally, the parenthetical subscript w is an integer typically in the range from 0 to 2.

In another aspect, the invention provides a method of inhibiting or disrupting the interaction between an alpha helix of a first protein and the alpha helix binding pocket of a second protein wherein the second protein with a compound of the present invention.

In another aspect, the invention provides a pharmaceutical composition which includes one or more compounds of the present invention and a pharmaceutically acceptable excipient.

### BRIEF DESCRIPTION OF THE DRAWINGS

FIG. 1 is a graphic representation of a CAVEAT search using Cα-Cβ bonds of F19, W23 and L26.

FIG. 2 is a graphic representation of scaffold mimicry of i, i-4 and i-7 alpha helix.

FIG. 3 is an exemplary set of side chains that are appended to the scaffolds of the invention.

### DETAILED DESCRIPTION

Definitions

Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et a!. MOLECULAR CLONING: A LABORATORY MANUAL, 2d ed. (1989) Cold Spring Harbor Laboratory Press, Cold Spring Harbor, N.Y., which is incorporated herein by reference) which are provided throughout this document. The nomenclature used herein and the laboratory procedures in analytical chemistry, organic synthetic chemistry, and pharmaceutical formulation described below are those well known and commonly employed in the art. Standard techniques are used for chemical syntheses, chemical analyses, pharmaceutical formulation and delivery, and treatment of patients.

“Analyte”, as used herein means any compound or molecule of interest for which a diagnostic test is desired. An analyte can be, for example, a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc., without limitation.

“Moiety” refers to the radical of a molecule that is attached to another moiety. It is within the scope of the present invention to include one or more sites that are cleaved by the action of a “cleavage agent” other than an enzyme. Cleavage agents include, but are not limited to, acids, bases, light (e.g., nitrobenzyl derivatives, phenacyl groups, benzoin esters), and heat. Many cleaveable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J Biochem., 155: 141-147 (1986); Park et al., J. Biol. Chem., 261: 205-210 (1986); Browning et al., J. Immunol., 143: 1859-1867 (1989).

The symbol [symbol] , whether utilized as a bond or displayed perpendicular to a bond indicates the point at which the displayed moiety is attached to the remainder of the molecule, solid support, etc.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stercoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY ₅TH ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (125I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents which would result from writing the structure from right to left, e.g., —CH₂O— is intended to also recite —OCH₂—; —NHS(O)₂— is also intended to represent. —S(O)₂HN—, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ′O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —N—RSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen-carbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

“Peptide” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

As used herein, “amino acid” refers to a group of water-soluble compounds that possess both a carboxyl and an amino group attached to the same carbon atom. Amino acids can be represented by the general formula NH₂—CHR—COOH where R may be hydrogen or an organic group, which may be nonpolar, basic acidic, or polar. As used herein, “amino acid” refers to both the amino acid radical and the non-radical free amino acid.

“Protein” refers to compounds comprising at least one polypeptide.

“Alpha helix” refers to a form of secondary structure in a protein in which the polypeptide chain is coiled into a helix. Typically, the helical structure is held in place by intramolecular hydrogen bonds.

As used herein, “disrupting” an interaction between a first protein and a second protein refers to the process of perturbing one or more covalent or non-covalent bonding interactions between the first and the second protein. Covalent bonding interactions between proteins include, for example, disulfide bonds, ester bonds, amide bonds and the like. Non-covalent bonding interactions between proteins include, for example, hydrophobic interactions, van der Waals interactions, ionic interactions, hydrogen bonding interactions and the like.

“Inhibiting” an interaction between a first protein and a second protein refers to the process of lowering the overall ability of the two proteins to bind or associate.

### Calculational Method

The crystal structure of the p53 peptide bound to the MDM2 N-terminal domain reveals a large hydrophobic pocket occupied by amino acids F19, W23, and L26 of p53. The inventors have used structure-based computational methods to design scaffolds for combinatorial libraries that produce organic molecules that bind to MDM2 at this hydrophobic binding site. Each scaffold has been designed to present side chains in the same manner that p53 presents those of F19, W23, and L26.

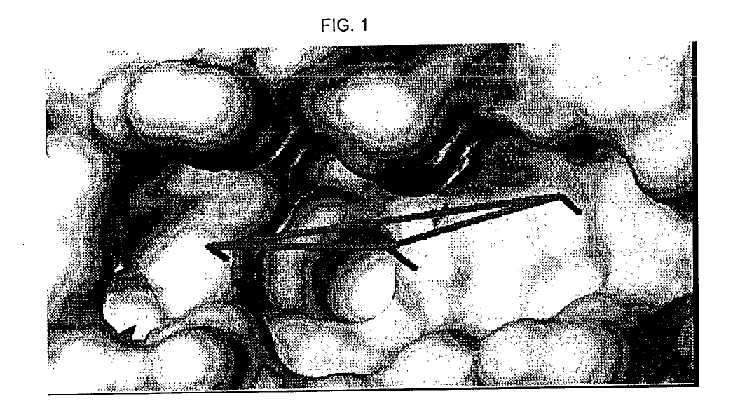

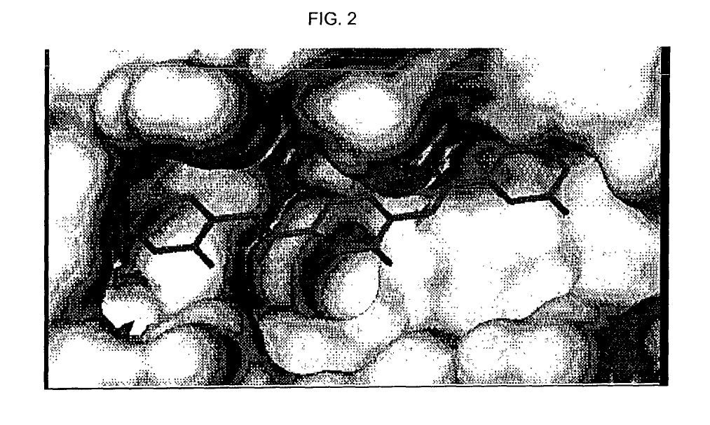

The program CAVEAT was used to find small molecules in the available chemical directories and other databases that contain bonds having approximately the same geometrical relationship as the Cα-Cβ bonds of F19, W23, and L26 (FIG. 1). The CAVEAT leads were then filtered and evaluated with DOCK to select for semi-rigid scaffolds that fit in the binding site of MDM2. Synthetically accessible scaffolds were chosen for library synthesis, and further DOCKing was performed to maximize the shape and chemical complementarity of the side chains to be attached to the scaffold (FIG. 2).

### Compounds

The present invention provides a family of compounds that inhibit or disrupt protein-protein interactions. In a first aspect, the invention provides a compound having a formula selected from:

A-L-B-L¹-A¹

and

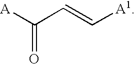

In this first aspect, substituent A is typically selected from the group:

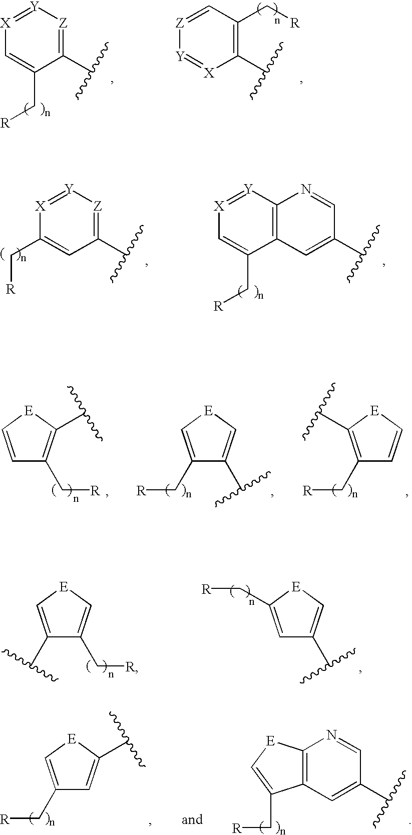

Substituent A¹ is typically selected from the group:

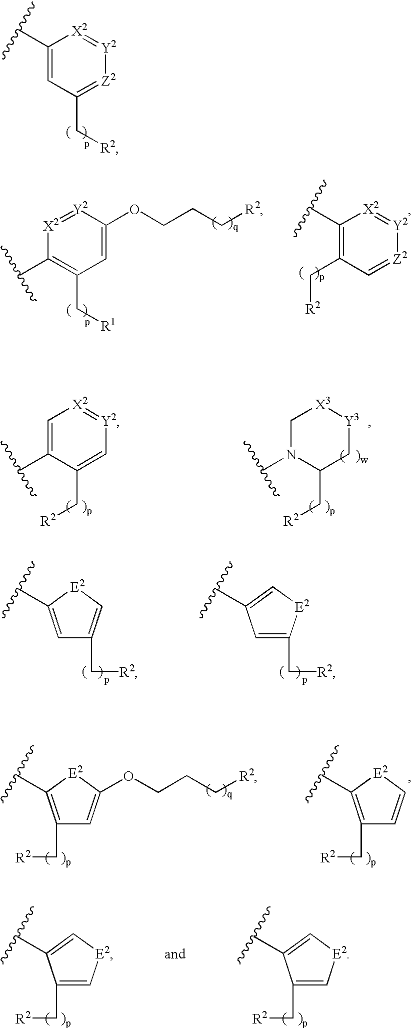

The core moiety, B, is typically selected from the group:

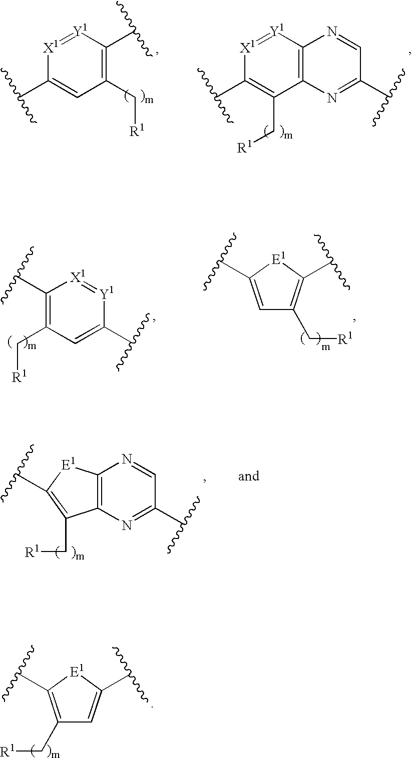

The linker moieties L and L¹ are typically selected from the group:

—N═N—, —CH₂—CH₂—, —C═C—, —CH₂—CH₂—, —CH₂—S—, —CH₂—NH—, —NH—CH₂,

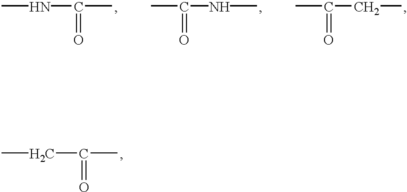

and a single bond.

In the above structures of the first aspect, the side group substituents R, R¹, and R² are typically selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl.

The ring moieties X, X¹, X², Y, Y¹, Y², Z, Z¹, and Z² are typically selected from —N— and —CH—. The ring moieties X³, Y³, E, E¹, and E² are typically selected from —NH—, —CH₂—, —S—, and —O—.

The parenthetical subscripts n, m, p and q are integers typically in the range from 0 to 4. The parenthetical subscript w is an integer typically in the range from 0 to 2.

One of skill in the art will immediately recognize that the individually labeled moieties, substituents, and parenthetical subscripts described above may be independently selected from the corresponding groups. For example, R may be hydrogen while R¹ is a substituted or unsubstituted alkyl and R² is a substituted or unsubstituted heteroaryl. Unless otherwise noted, all individually labeled moieties, substituents, and parenthetical subscripts presented herein may be individually selected from the corresponding groups.

In an exemplary embodiment of the first aspect, the compound has a formula typically selected from the group:

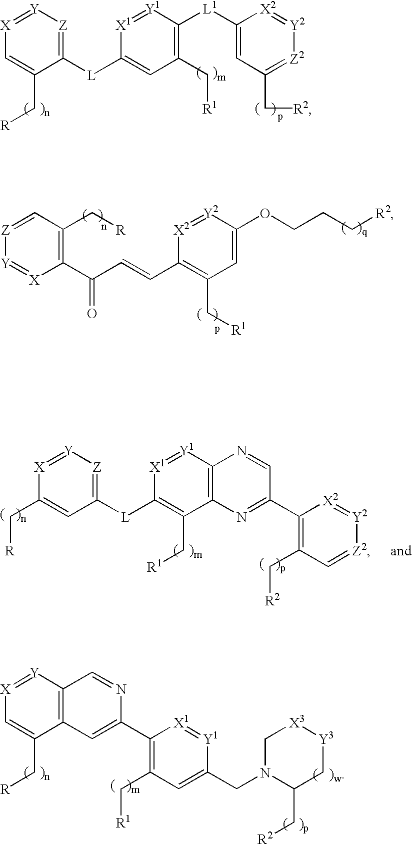

In this exemplary embodiment, the linker moieties L and L¹ are typically selected from the group:

—N═N—, —CH₂—CH₂—, —C═C—, —CH₂—CH₂—, —CH₂—S—, —CH₂—NH—, —NH—CH₂—,

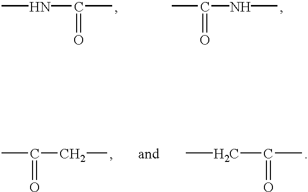

The side group substituents R, R¹, and R² are typically selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl.

The ring moieties X, X¹, X², Y, Y¹, Y², Z, Z¹, and Z² are typically selected from —N— and —CH—. The ring moieties X³ and Y³ are typically selected from —NH—, —CH₂—, —S—, and —O—.

The parenthetical subscripts n, m, p and q are integers typically in the range from 0 to 4. The parenthetical subscript w is an integer typically in the range from 0 to 2.

As disclosed above, the side group substituents, R, R¹, and R² are typically selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl.

In a further exemplary embodiment, the substituted or unsubstituted heteroaryl referred to in the previous paragraph is typically selected from substituted or unsubstituted pyridyl, substituted or unsubstituted pyrrolyl, substituted or unsubstituted imidizolyl, substituted or unsubstituted oxazolyl, substituted or unsubstituted thiazolyl, substituted or unsubstituted indolyl, substituted or unsubstituted isoquinolyl, and substituted or unsubstituted purinyl.

In another exemplary embodiment, the substituted or unsubstituted aryl is selected from substituted or unsubstituted phenyl, substituted or unsubstituted naphthyl, and substituted or unsubstituted biphenylmethyl.

In another exemplary embodiment, the substituted or unsubstituted heterocycloalkyl is selected from substituted or unsubstituted pyrrolidinyl, substituted or unsubstituted morpholino, substituted or unsubstituted piperidinyl, and substituted or unsubstituted tetrahydropyranyl.

In another exemplary embodiment, the substituted or unsubstituted cycloalkyl is selected from substituted or unsubstituted cyclopentyl, and substituted or unsubstituted cyclohexyl.

In another exemplary embodiment, the substituted or unsubstituted alkyl is selected from substituted or unsubstituted methyl, substituted or unsubstituted ethyl, substituted or unsubstituted propyl, substituted or unsubstituted butyl, and substituted or unsubstituted pentyl.

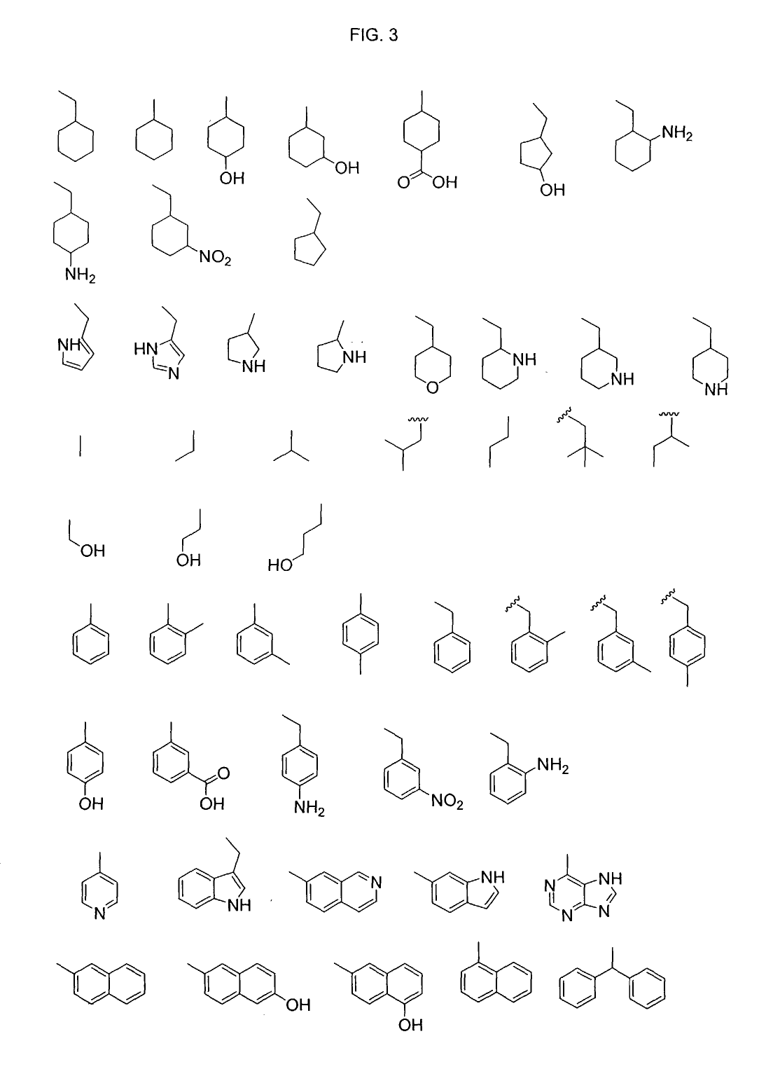

In yet a further exemplary embodiment, the R—(CH₂)ₙ, R¹—(CH₂)ₘ and R²—(CH₂)ₚ are members independently selected from the moieties represented in FIG. 3.

### Exemplary Syntheses

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention, it is not intended to define the scope of reactions or reaction sequences that are usefui in preparing the compounds of the present invention.

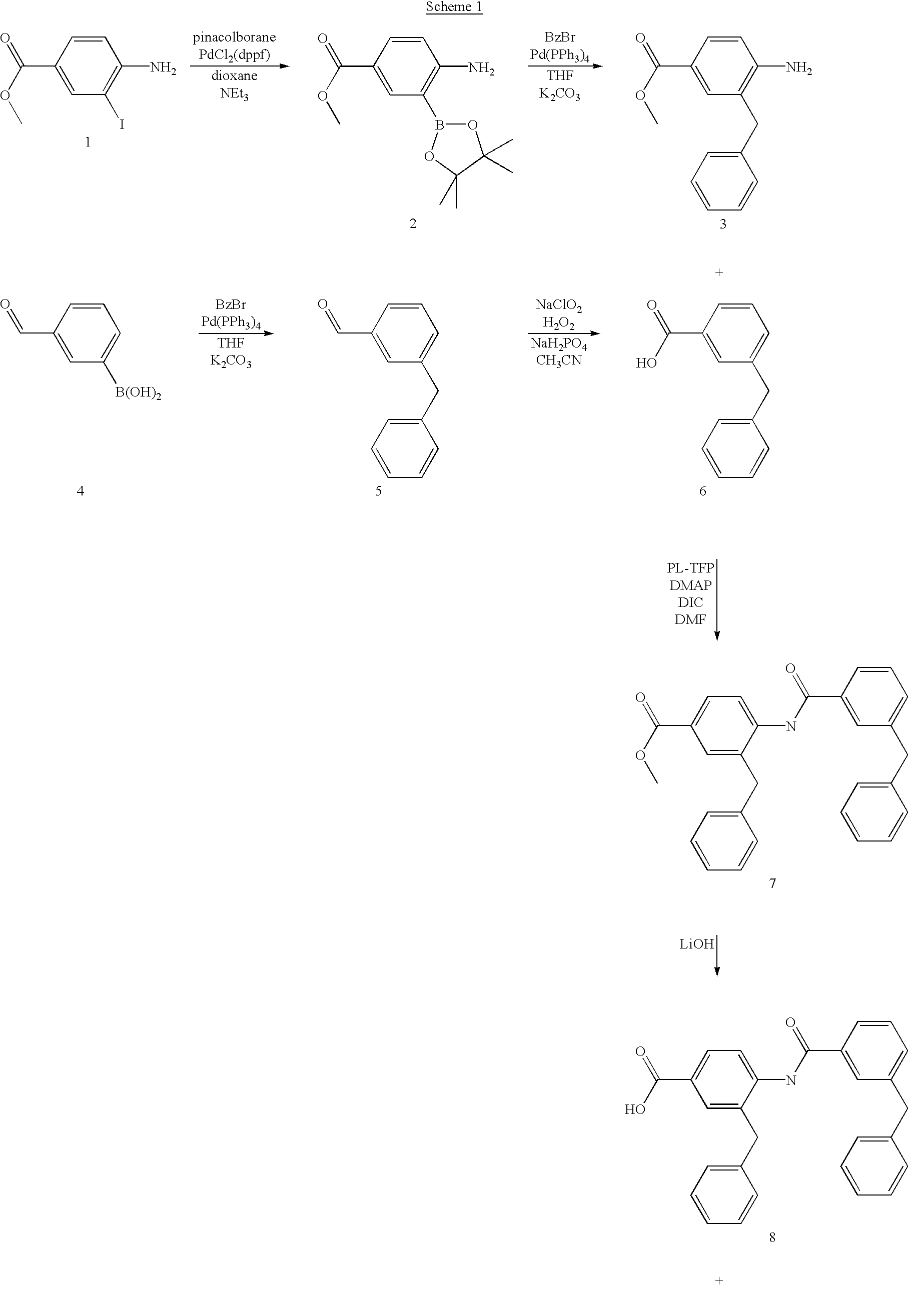

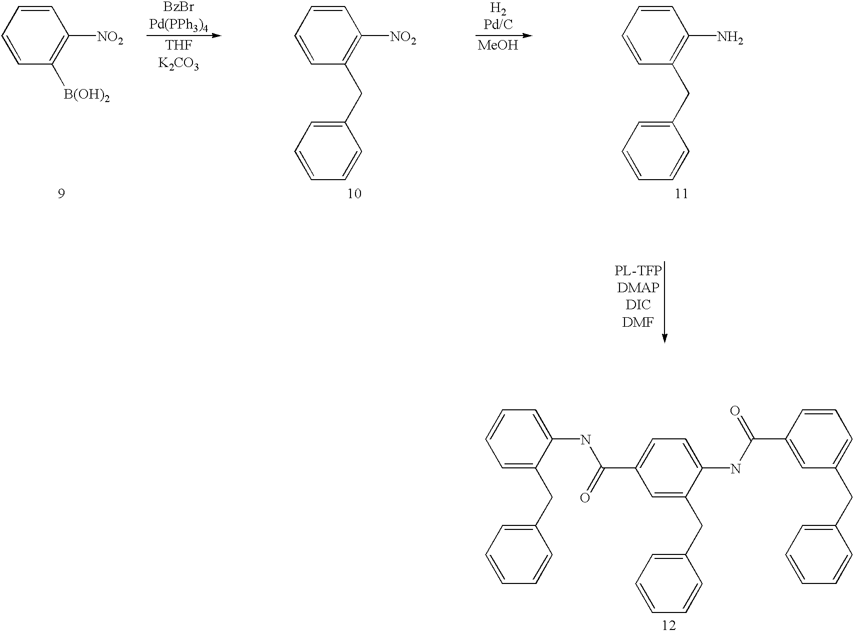

In the first exemplary synthesis (Scheme 1), a triaryl diamide compound 12 is synthesized. The diamide compound 12 is assembled by linking together three monomer subunits, 3, 6 and 11 using a catch and release methodology as described below.

The first monomer subunit, the methyl-amino-benzylbenzoate 3, is synthesized by first combining pinacolborane with the amino-benzoate 1 in the presence of a palladium catalyst to form the corresponding amino-dioxaborolanylbenzoic acid methyl ester 2.

Next, Suzuki coupling reaction conditions are employed to displace the dioxaborolanyl with a benzyl substituent to form 3 (Miyaura et al., Chem. Rev. 95: 2457-2483 (1995)). Metal-catalyzed coupling reactions are well known in the art. One skilled in the art will recognize that a variety of nucleophiles may be used in these coupling reactions including, but not limited to, RMgX, RZnX, RZr, ROH, and RSH, wherein R is a substituent group as defined above (see page 11) and X is a halide. A person of skill in the art would recognize that a variety of R groups may be introduced in the current exemplary synthesis.

Suzuki coupling conditions are again employed to prepare the second monomer subunit, the benzyl-benzoic acid 6. More specifically, formylphenyl boronic acid 4 is contacted with benzylbromide in the presence of a palladium catalyst to form the benzyl-benzaldehyde 5. Next, 5 is oxidized to the corresponding carboxylic acid 6 using sodium chlorite and peroxide in solution.

The formation of the amide bond between 3 and 6 to yield the amide-carboxylic acids 7 is performed using a catch and release methodology with a tetrafluorophenol (TFP) resin (Salvino et al., Journal of Combinatorial Chemistry 2: 691-697 (2001)). The resin is used to form an active ester polymer from the benzoic acid. The resin facilitates purification and handling of the active ester. Methods for forming amide bonds, both in the solid phase and in the solution phase, are well known in the art (see e.g., Stewart et al., Solid Phase Peptide Synthesis, 2nd Ed., 1984). One skilled in the art will immediately recognize that a variety of solid phase and solution phase amide bond formation methods are of use in the current invention. After amide bond formation, 7 is saponified to the corresponding carboxylic acid 8 with lithium hydroxide

The third monomer subunit, the benzyl-phenylamine 11, is synthesized by first displacing the boronic acid substituent of the nitrophenylboronic acid 9 with a benzyl moiety to form the benzylnitrobenzene 10. Hydrogenation of 10 with a palladium catalyst yields the benzyl-phenylamine 11.

Finally, an amide bond is formed between 8 and 11 using TFP resin as described above to yield the diamide 12.

In another exemplary synthesis, a chalcone compound with variable side chain groups (R₁, R₂ and R₃) 24 is synthesized (Scheme 2). A variety of chemical moieties are useful as the side chain groups R₁, R₂ and R₃. Examples of side chain groups include, but are not limited to, the substituents presented in FIG. 3.

The synthesis begins by exposing p-hydroxybenzaldehyde 13 to an organic bromination reagent that affords the corresponding bromide 14. Next, 14 is protected as the tert-butyldimethylsilyl ether and converted into the aryl boronic acid by palladium catalyzed cross coupling with a diborane to give the protected aryl boronic acid 15. The protected 15 is then allowed to react with the appropriate aryl bromide under Suzuki reaction conditions resulting in the formation of the first monomer 18, which contains the variable group R₂.

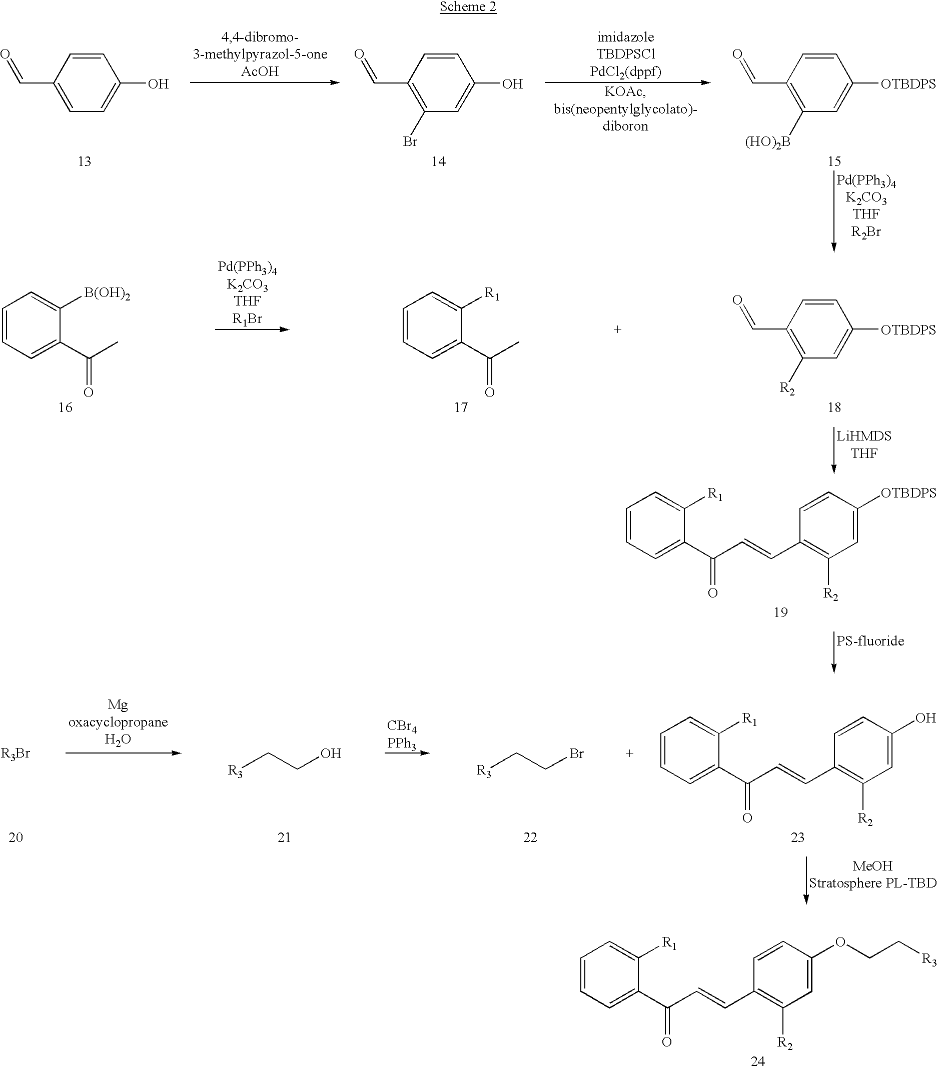

The Suzuki reaction is again used to introduce a variable side chain group to the aryl boronic acid ketone 16 to yield the second monomer 17, which contains the variable group R₁. Sequential treatment of monomers 17 and 18 with lithium hexamethyldisilylazide provides the enone 19 (Daskiewicz et al., Tetrahedron Lett. 40: 7095-7098 (1999)). The enone intermediate 19 is deprotected with polymer supported fluoride (Cardillo et al., Chem. Ind. (London) 16: 643-644 (1983)).

The third monomer 22 is prepared from appropriate bromide 20 Netherton et al., Journal of the American Chemical Society 123: 10099-10100 (2001)). Exposure of 20 to magnesium with a trace of iodine forms the Grignard reagent that is trapped with gaseous oxirane to afford the variably substituted ethyl alcohol 21. Next, treatment of 21 with triphenylphosphine and carbon tetrabromide provides the variably substituted ethyl bromide 22 (Madema et al., Journal of the American Chemical Society 123: 10423-10424 (2001)). Finally, 22 is introduced to 23 in the presence of a polymer supported DBU analog to from the variably substituted chalcone 24 (Xu et al., Tetrahedron Lett. 38: 7337-7340 (1997)).

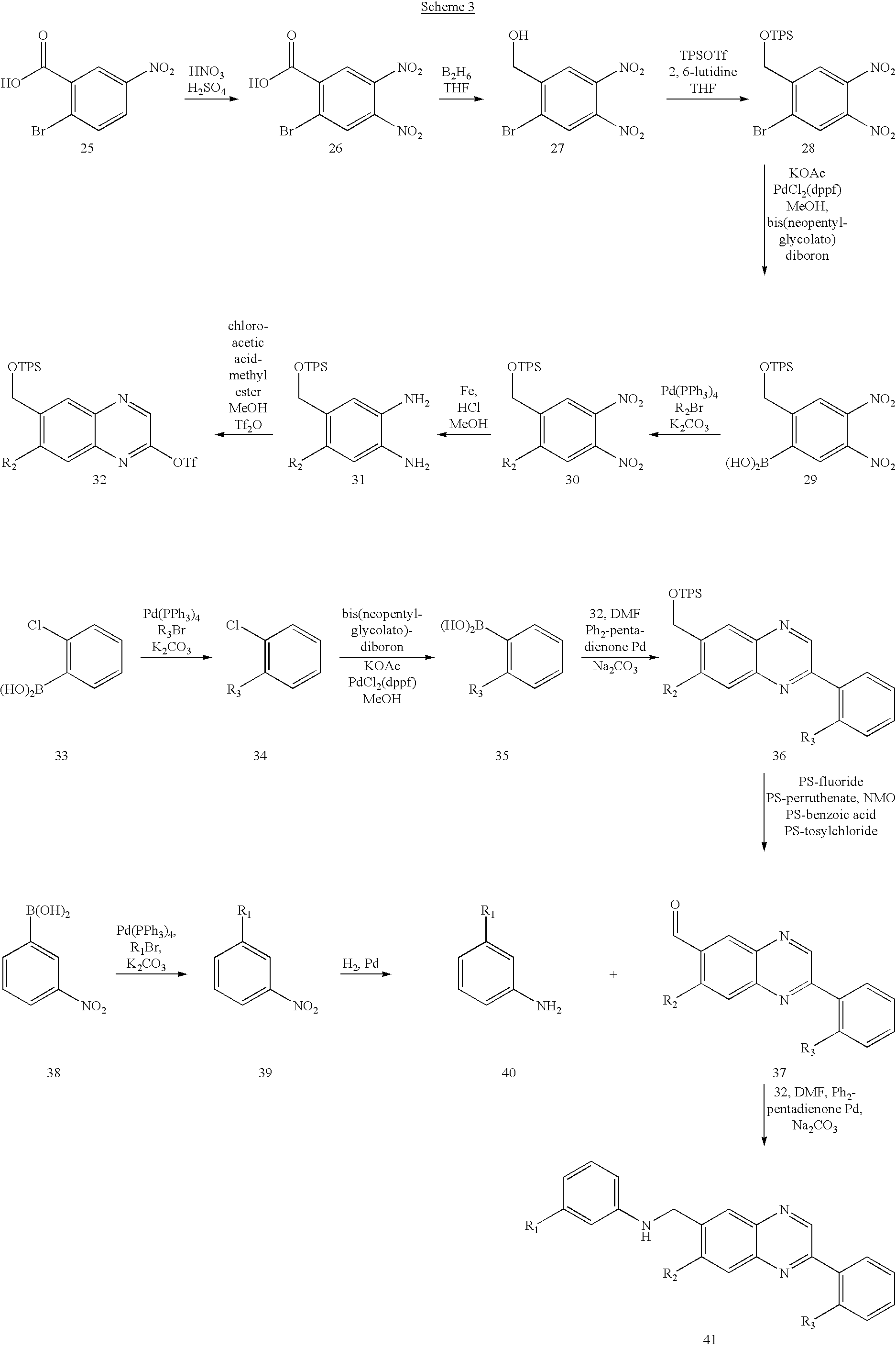

In another exemplary synthesis, the variably labeled quinoxaline 41 is synthesized (Scheme 3). The assembly of the first variably labeled monomer 28 starts with bromonitrobenzoic acid 25. Nitration of 25 affords the tetrasubstituted compound 26 (Goldstein et al, Helv. Chim. Acta 26: 173-181 (1943)). Exposure of 26 to diborane in THF leads to selective reduction of the carboxylic acid without accompanying reduction of the nitro groups, thus producing 27. The benzyl alcohol 27 is protected as the triphenylsilyl ether 28, which is then converted into the boronic acid 29 by a palladium catalyzed reaction with a diborane (Miyaura et al., Tetrahedron Lett 27: 6369-6372 (1986)) The variable side chain group, R₂, is introduced using Suzuki coupling conditions appropriate bromide to give 30 (Miyaura et al., Chem. Rev. 95: 2457-2483 (1995); Netherton et al., Journal of the American Chemical Society 123: 10099-10100 (2001)). Reduction of the two nitro groups with iron and hydrochloric acid provide the diamine 31 (Goldstein et al., Helv. Chim. Acta 26: 173-181 (1943)). Finally, the diamine 31 is condensed with chloroacetic acid under dehydrative conditions followed by conversion to the triflate with triflic anhydride providing 32 (Campaigne et al., J. Heterocycl. Chem. 20: 623-628 (1983)).

The second variably substituted monomer 35 is generated from chloroboronic acid 33 by Suzuki coupling giving 34. The chloride 34 is converted to the boronic acid 35 by palladium catalyzed cross coupling with a diborane (Ishiyama et al., J. Org. Chem. 60: 7508-7510 (1995)).

The third variably substituted monomer is produced by subjecting 38 to Suzuki coupling with the appropriate bromide to afford the variably substituted nitroarene 39. Reduction of 39 with palladium on carbon provides the desired aniline 40.

Suzuki coupling between 32 and 35 provides the arylquinoxaline 36 (Hersperger et al., J. Med. Chem. 43: 675-682 (2000)). The silyl protecting group of 36 is removed using polymer supported fluoride. The resulting alcohol is oxidized using polymer supported perruthenate to provide the aldehyde 37. The remaining morpholines are scavenged using supported benzoic acid. Supported tosyl chloride is used to scavenge any remaining oxides and alcohols. (Cardillo et al, Chem. Ind. (London) 16: 643-644 (1983); Hinzen et al., J. Chem. Soc., Perkin Trans. 1: 1907-1908 (1997)). Finally, reductive amination of 37 with 40 provides the variably substituted quinoxaline 41.

In another exemplary synthesis the arylisoquinoline 57 is synthesized (Scheme 4). The generation of the first variably substituted monomer 46 begins with the bromophenol 42. Hydrolysis of the amide function to an acid using acidic conditions provides 43. Next, the acid functionality is converted into an aldehyde by first protecting the phenol as the tert-butyldimethylsilyl ether (Corey et al., J. Amer. Chem. Soc. 94: 6190-6191 (1972)). The acid is reduced with an aluminum reducing agent in dimethyl ether and immediately reoxidized to the aldehyde using tetrapropylammonium perruthenate to give 44 (Griffith et al., J. Chem. Soc., Chem. Commun. 21: 1625-1627 (1987); Gao et al., J. Org. Chem. 53: 4081-4084 (1988)) Finally, the variable side chain group R₁ is introduced by conversion of the bromide to the boronic acid followed by Suzuki cross coupling to give the aldehyde 45. The silyl protecting group is removed with polymer-supported fluoride and converted into the aryl triflate 46.

The generation of the second variably substituted monomer 53 begins with chlorophenol 47. Protection of the aldehyde as the cyclic acetal followed by triflation of the phenol provides the aryl triflate 48 (Showler et al., Chem. Rev. 67: 427-440 (1967)), which is subjected to Sonogashira coupling conditions to afford protected acetylene 49 (Zhang et al., J. Org. Chem. 65: 7977-7983(2000). Next, the variable side chain group R₂ is introduced by Suzuki coupling with the R₂ bromide followed by removal of the silyl protecting group with polymer supported fluoride to give 50.

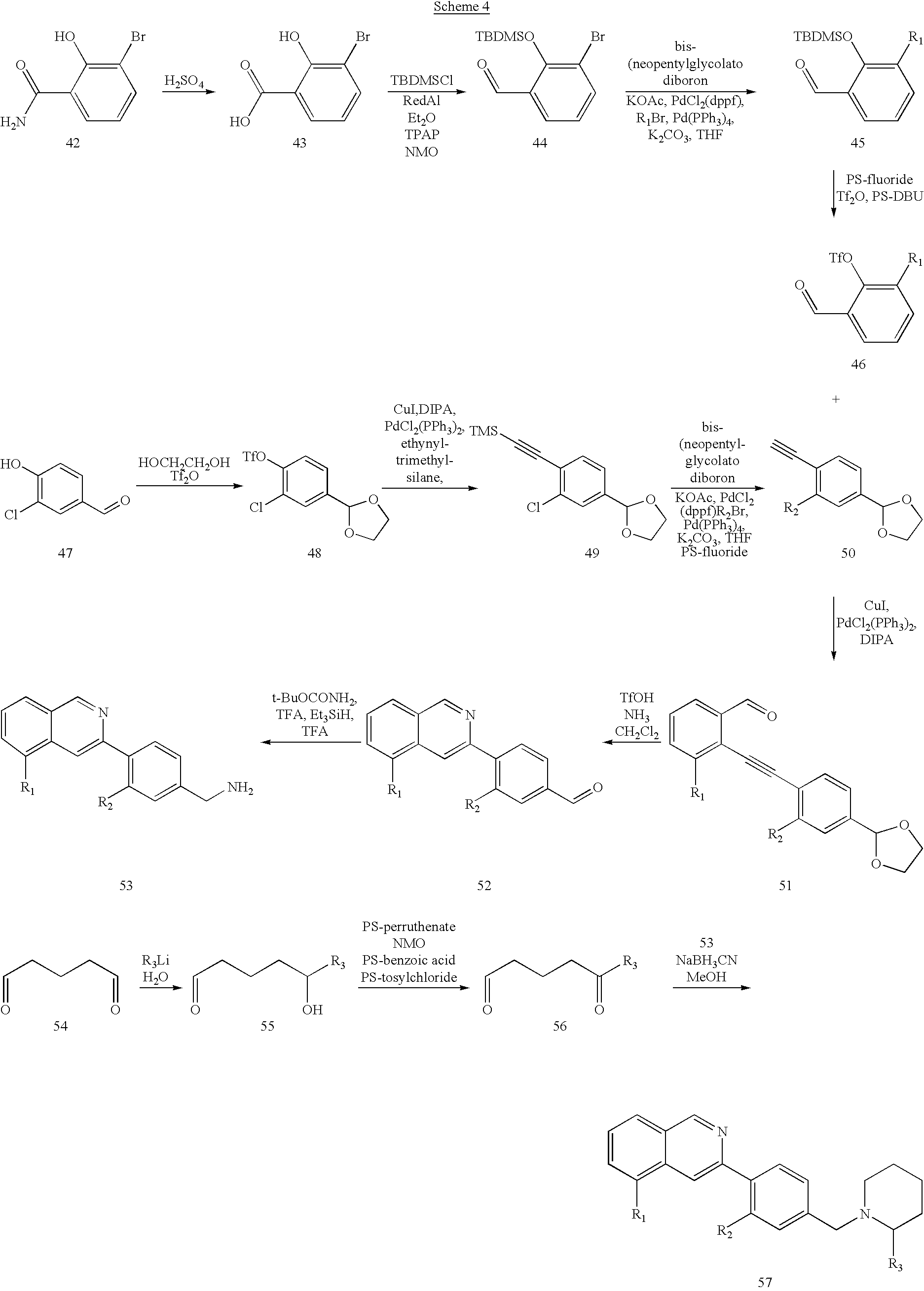

The synthesis of the third variably substituted monomer 56 begins by allowing the dialdehyde 54 to react with one equivalent of the appropriate Grignard or lithium reagents to give the variably substituted alcohol 55. Oxidation of 55 using polymer-supported perruthenate with the normal scavenger resin cleanup procedure provides 56 (Hinzen et al., J. Chem. Soc., Perkin Trans. 1: 1907-1908 (1997)).

Next, the trifiate 46 and acetylene 50 are allowed to react under Sonogashira-Hagiwara conditions, which affords the diaryl acetylene 51 (Tovar et al., J. Org. Chem. 64: 6499-6504 (1999)). Treatment with ammonium triflate in methylene chloride results in ring closure to give the quinoline 52 (Tovar et al., J. Org. Chem. 64: 6499-6504 (1999)). Finally, the aldehyde function is reductively aminated using conditions developed to give the primary amine 53 (Dube et al., Tetrahedron Lett. 40: 2295-2298 (1999). Finally, 56 is introduced by double reductive amination of amine 53 with the aldehyde-ketone 56 to give the substituted quinoline 57 (Barili et al., Tetrahedron 53: 3407-3416 (1997).

### Methods

The present invention also provides methods of inhibiting or disrupting the interaction between two proteins. In a second aspect, the invention provides a method of inhibiting or disrupting the interaction between an alpha helix of a first protein and the alpha helix binding pocket of a second protein. In this second aspect, the method includes the step of contacting the second protein with a compound of the present invention. Compounds of the present invention are disclosed above.

In an exemplary embodiment, the method includes contacting a complex between the first and second protein with a protein of the current invention.

Protein-protein interactions involving an alpha helix of a first protein and a alpha helix pocket of a second protein are well known in the art. Without being limited by any particular theory, the mechanism of binding appears to involve the fitting of the hydrophobic face of a small amphipathic alpha helix of one protein into a well-defined pocket on another protein during their binding to one another. Examples of such interactions include, but are not limited to heterotrimeric G protein alpha subunit with adenylyl cyclase (Sunahara et al., Science 278: 1943-1947 (1997); Tesmer et al., Science 278: 1907-1916 (1997)), the interaction of hTRβ1 and GRIP1 (Feng et al., Science 280:1747-1749 (1998)), the binding of VP16 to hTAFII31 (Uesugi et al., Science 277: 1310-1313 (1997)), and the binding of the MDM2 oncoprotein to p53 (Kussie, et al., Science 274: 948-953 (1996)).

In an exemplary embodiment, the invention provides a method of disrupting or inhibiting the interaction between p53 and MDM2. The method includes contacting an MDM2 polypeptide comprising an alpha helix binding pocket with a compound of the present invention. In another exemplary embodiment, the method includes contacting a complex between the first and second protein with a protein of the current invention.

Methods of determining the inhibition or disruption of protein-protein interactions are well known in the art. One skilled in the art will recognize that a variety of known methods may be useful in the present invention. Such methods include, but are not limited to, gel shift assays, GST-pull down competition assays, immunoprecipitation assays, equilibrium dialysis assays, surface plasmon resonance binding assays, cellular based reporter gene assays and the like.

In an exemplary embodiment, the inhibition or disruption of the p53-MDM2 interaction is measured using fluorescence anisotropy competition assays (Owicki et al., J Biomol Screen 5: 297-306 (2000)).

In a further exemplary embodiment, the fluorescence anisotropy competition assay employs a fluorescently labeled p53 peptide and a recombinant (His)6-tagged MDM2 protein expressed heterologously in E. coli (Bottger et al., Curr Biol 7: 860-869 (1997); Kussie et al., Science 274: 948-953 (1996)). A compound of the present invention is added to disrupt or inhibit the interaction between the fluorescently labeled p53 peptide and the recombinant MDM2 protein. Thus, the degree of disruption or inhibition is determined by measuring the change in the fluorescence parameter using fluorescence anisotropy (FA).

In another exemplary embodiment, the inhibition or disruption of the p53-MDM2 interaction is measured by assaying for the suppression of MDM2 activity in cells (see Woods et al., Mol Cell Biol 17: 5598-611 (1997); Ries et al., Cell 103: 321-30 (2000)). Elevated levels of MDM2 protein suppresses the induction of p53 activity in response to DNA damage agents such as γ-irradiation or adriamycin (Ries et al., Cell 103: 321-30 (2000)). In addition, MDM2 gene transcription is induced by the Raf→MEK→MAP kinase pathway through Ets and AP-1 transcription factors. In the present embodiment, a p53 responsive promoter is used to drive a reporter gene and faithfully reveal the levels of p53 activity as well as its regulation by Raf induced MDM2. Thus, inhibition or disruption of the MDM2-p53 interaction is measured by the degree of suppression of reporter gene expression.

In a further exemplary embodiment, the p53 responsive reporter gene comprises the human placental secreted alkaline phosphatase (pSEAP). This reporter is heat resistant and quite stable (Durocher et al., Anal Biochem 284: 316-26 (2000)). These properties allow kinetic assays to be carried out because the media can be periodically removed and replaced without disturbing the cells. To reduce background, the media is heated to 65° C. prior to adding a developing reagent, which inactivates the majority of alkaline phosphatases but not pSEAP. The pSEAP enzyme is developed chromophorically, fluorescently, or luminescently, depending on the desired dynamic range of the assay.

In a still further exemplary embodiment, the cellular system stably expresses a fusion protein between the protein kinase domain of Raf-1 and the hormone binding of the estrogen receptor (ΔRaf-1:ER) in an NIH-3T3 cell background. The fusion protein is selectively activated by treatment of cells expressing the fusion protein with 4-hydroxytamoxifen (4-HT) (see Woods et al., Mol Cell Biol 17: 5598-611 (1997); Ries et al., Cell 103: 321-30 (2000)). The cellular system also stably expresses the p53 responsive reporter. The production of this stable cell line reduces variability induced in the assay by relative differences in transfection efficiency. In this embodiment, cells are exposed to 4-HT to activate ΔRaf-1:ER for 24 hours leading to elevated expression of MDM2. The cells are then incubated with at lest one compound of the current invention and 4-HT for 4 hours to allow for inhibition or disruption of the MDM2-p53 interaction. The cells are then treated with adriamycin for a total time period of 8 hours to induce DNA damage. Media is collected every two hours following the addition of adriamycin. The media is assayed for reporter gene product activity (e.g. SEAP activity). Inhibitors of the MDM2-p53 interaction counteract the effects of activated Raf and lead to elevated reporter gene product activity in the media.

### Pharmaceutical Composition

The present invention also provides pharmaceutical compositions. In a third aspect, the invention provides a pharmaceutical composition which includes one or more compounds of the present invention and a pharmaceutically acceptable excipient. Compounds of the present invention are disclosed above. These compounds typically comprise the active component of the pharmaceutical compositions.

The compounds of the present invention can be prepared and administered in a wide variety of oral, parenteral and topical dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active component. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active component with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The dose administered to a patient, in the context of the present invention should be sufficient to effect a beneficial therapeutic response in the patient over time. The dose will be determined by the efficacy of the particular compound employed and the condition of the patient, as well as the body weight or surface area of the patient to be treated. The size of the dose also will be determined by the existence, nature, and extent of any adverse side-effects that accompany the administration of a particular compound in a particular patient.

The compound can also be introduced into an animal cell, preferably a mammalian cell, via a microparticles and liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell.

The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., PNAS 84:7851 (1987); Biochemistry 28:908 (1989)). When liposomes are endocytosed by a target cell, for example, they become destabilized and release their contents. This destabilization is termed fusogenesis. Dioleoylphosphatidyl-ethanolamine (DOPE) is the basis of many “fusogenic” systems.

Such liposomes typically comprise a compound of choice and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91\17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments of the present invention, it is desirable to target the liposomes of the invention using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., J. Biol. Chem., 265:16337-16342 (1990) and Leonetti et al., PNAS 87:2448-2451 (1990).

In determining the effective amount of the compound to be administered in the treatment or prophylaxis of conditions owing to HIV infection, the physician evaluates circulating plasma levels of the compound, compound toxicities, progression of the disease, and the production of viral resistance to the compound.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The quantity of active component in a unit dose preparation may be varied or adjusted from 0.1 mg to 10 g, more typically 1.0 mg to 1 g, most typically 10 mg to 500 mg, according to the particular application and the potency of the active component. The composition can, if desired, also contain other compatible therapeutic or diagnostic agents. Administration can be accomplished via single or divided doses.

### EXAMPLES

### Example 1

### Synthesis of 3-Benzyl-benzaldehyde 5

Under argon atmosphere: To a mixture of THF (12.5 mL) and 2M K₂CO₃ (5 mL, 10 mmol) were added 3-formylphenylboronic acid 4 (0.50 g, 3.3 mmol), benzyl bromide (0.36 mL, 3 mmol), and Pd(PPh₃)₄ (0.087 g, 0.075 mmol). Full conversion was reached after 16 h at 80° C. as indicated by TLC. The reaction was quenched with HCl and the aqueous phase was extracted with ether. Solvent was removed in vacuo from the combined organic layers. The crude material was purified by flash chromatography (silica gel, hexanes/ethyl acetate (12:1)) to give 0.5 g (80%) of the product. ¹H NMR (400 MHz, CDCl₃): 6=9.98 (s, 1H), δ=7.723 (m, 1H), δ=7.459 (d, J=5.6 Hz, 1H), δ=7.26 (m, 7H), δ=4.062 (s, 2H).

### Example 2

### Synthesis of 3-Benzyl-benzoic Acid 6

A solution of NaClO₂ (0.36g, 14 mmol) in water (4 mL) was added dropwise in 1 h to a stirred mixture of 3-benzyl-benzaldehyde 5 (0.39g, 2.0 mmol), NaH₂PO₄ (0.58g, mmol), and 35% H₂O₂ (1 mL, 10 mmol) in acetonitrile (15 mL) and water (7 mL), keeping the temperature below 10° C. using an ice bath. After the addition was complete, the ice bath was removed and the reaction proceeded to completion after 2 hours. Sodium sulfite was added to quench the reaction, and the solution was acidified with HCI. The organic phase was separated and dried in vacuo to afford 0.54 g (75%) of the product. ¹H NMR (400 MHz, CDCl₃): δ=7.95 (m, 2H), δ=7.41 (m, 2H), δ=7.25 (m, 5H), δ=6.65 (bs, 1H), δ=4.046 (s, 2H).

### Example 3

### Synthesis of 2-Benzyl-nitrobenzene 10

Under argon atmosphere: To a mixture of THF (12.5 mL) and 2M K₂CO₃ (5 mL, 10 mmol) were added 2-nitrophenylboronic acid 9 (0.55 g, 3.3 mmol), benzyl bromide (0.36 mL, 3 mmol), and Pd(PPh₃)₄ (0.087 g, 0.075 mmol). Full conversion was reached after 16 h at 80° C. as indicated by TLC. The reaction was quenched with HCl and the aqueous phase was extracted with ether. Solvent was removed in vacuo from the combined organic layers. The crude material was purified by flash chromatography)siica gel, hexanes/ethyl acetate (12:1)) to give 0.22 g (33%) of the product. ¹sH NMR (400 MHz, CDCl₃): δ=7.932 (dd, J=8,1.4 Hz, 1H), δ=7.512(td, J=7.6, 1.2 Hz, 1H), δ=7.375 (td, J=7.6, 1.2 Hz, 1H), δ=7.4 (m, 6H), δ=4.312 (s, 2H).

### Example 4

### Synthesis of 2-Benzyl-phenylamine 11

Under hydrogen atmosphere: 10% palladium on carbon (20 mg, 50% wet) was added to a solution of 2-benzyl-nitrobenzene 10 (0.22 g, 1 mmol) in MeOH (15 mL). Full conversion was reached after 1 h, as indicated by TLC. The mixture was filtered and the solvent was removed in vacuo to afford 0.16 g (87%) of the product. ¹H NMR (400 MHz, CDCl₃): δ=7.4 (m, 6H), δ=6.768 (td, J=7.6, 1.2 Hz, 1H), δ=6.678 (d, J=8 Hz), δ=3.908 (s, 2H), δ=3.5 (bs, 2H).

### Example 5

### Synthesis of 4-Amino-3-(4,4,5,5-tetramethyl-(1,3.2)dioxaborolan-2-yl)-benzoic acid methyl ester 2

Under argon atmosphere: To a mixture of methyl 4-amino-3-iodo-benzoate 1 (2.27 g, 8.19 mmol) in 1,4-dioxane (20 ml), triethylamine (4.6 ml, 33 mmol) and PdCl₂(dppf) (0.30 g, 0.4 mmol), pinacolborane (3.6 ml, 25 mmol) was added dropwise at rt. Full conversion was reached after 8 h at 80° C. as indicated by TLC. The reaction was slowly quenched with sat. NH₄Cl (aq) and the aqueous phase was extracted with diethyl ether. After drying over MgSO₄, the solution was filtered over a patch of silica. Subsequently the silica was washed with methylene chloride. Concentration of the solution in vacuo gave 0.49 g of a mixture of the product and methyl-4-aminobenzoate. ¹H NMR (400 MHz, CDCl₃): 6=8.310 (d, J=2 Hz, 1H), 6=7.888 (d, J=2.4 Hz, 1H), 6=6.551 (d, J=8.8 Hz, 1H), 6=3.844 (s, 3H), 6=5.184 (bs, 2H), 6=1.346 (s, 12H).

### Example 6

### Synthesis of Methyl 4-amino-3-benzyl-benzoate 3

Under argon atmosphere: To a mixture of THF (8 mL) and 2M K₂CO₃ (1.6 mL, 10 mmol) were added crude 4-amino-3-(4,4,5,5-tetramethyl-(1,3,2)dioxaborolan-2-yl)-benzoic acid methyl ester 2 (0.49 g, 1.8 mmol), benzyl bromide (0.40 mL, 3.6 mmol), and Pd(PPh₃)₄ (0.050 g, 0.043 mmol). Full conversion was reached after 16h at 80° C. as indicated by TLC. The reaction was quenched with HCl and the aqueous phase was extracted with ether. Solvent was removed in vacuo from the combined organic layers. The crude material was purified by flash chromatography (silica gel, dichloromethane/hexanes (5:1)) to give 0.1 g (20%) of the product. ¹H NMR (400 MHz, CDCl₃): δ=7.81 (m, 2H), δ=7.2 (m, 5H), δ=6.634 (d, J=8.4 Hz, 1H), δ=3.930 (s, 2H), δ=3.898 (bs, 2H), δ=3.860 (s, 3H).

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

What is claimed is:

1. A compound selected from:
A-L-B-L¹-A¹
and 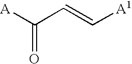
in which
A is a member selected from: 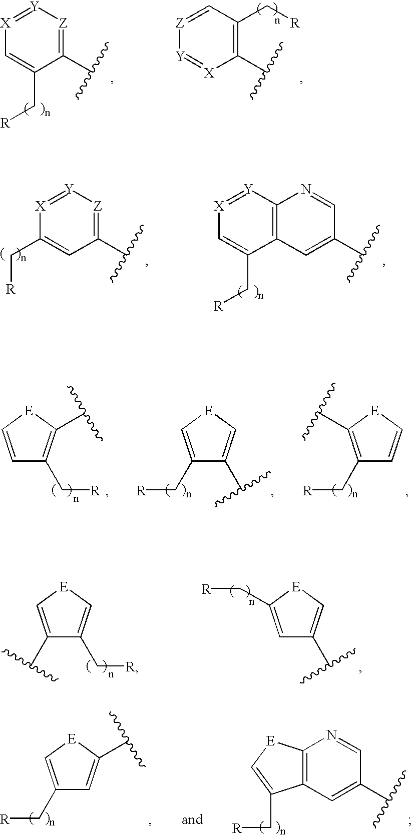
B is a member selected from: 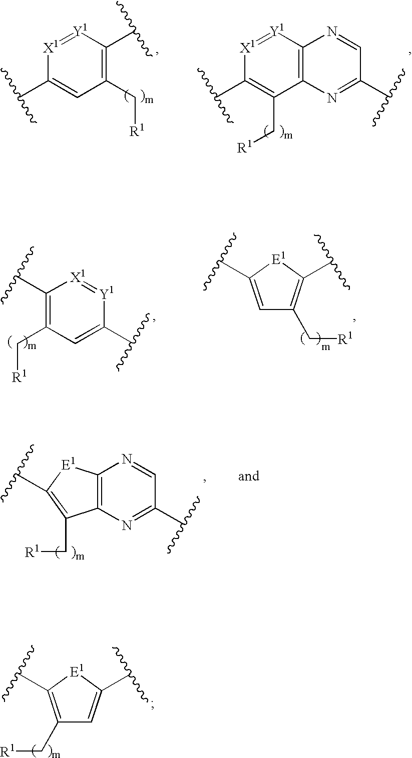
A¹ is a member selected from: 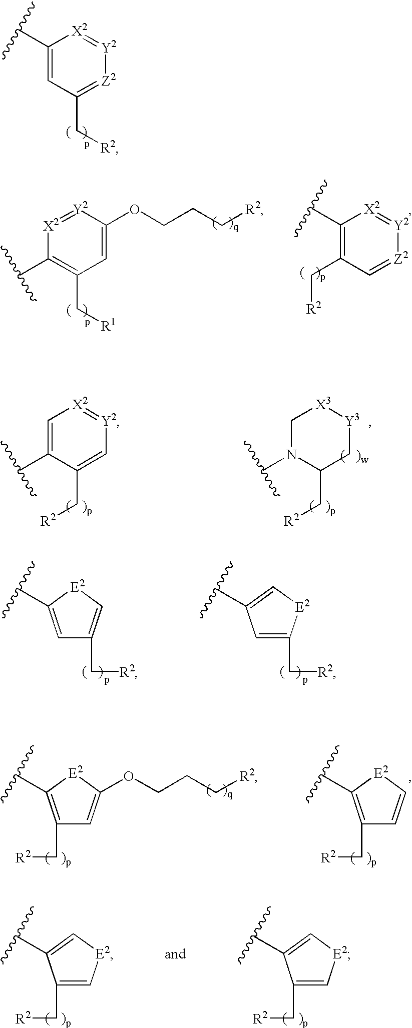
R, R′, and R² are members independently selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl;
L and L¹ are members independently selected from:
—N═N—, —CH₂—CH₂—, —C═C—, —CH₂—CH₂—, —CH₂—S—, —CH₂—NH—, —NH—CH₂, 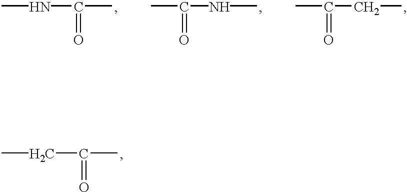
and a single bond;
X, X¹, X², Y, Y¹, Y², Z, Z¹, and Z² are members independently selected from:
—N— and —CH—;
X³, Y³, E, E¹, and E²are members independently selected from:
—NH—, —CH₂—, —S—, and —O—;
E, E¹, and E² members independently selected from:
—NH—, —CH₂—, —S—, and —O—;
n, m, p and q are integers independently selected from 0 to 4; and
w is an integer from 0 to 2.

2. A compound according to claim 1, having a formula which is a member selected from: 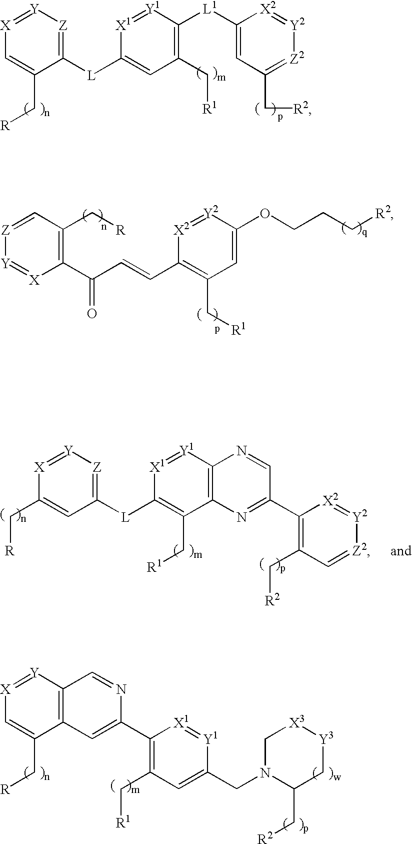
in which
L and L¹ are members independently selected from:
—N═N—, —CH₂—CH₂—, —C═C—, —CH₂—CH₂—, —CH₂—S—, —CH₂—NH—,
—NH—CH₂—, 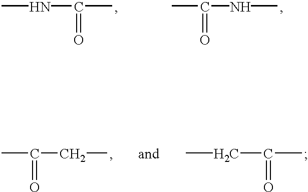
X, X¹, X², Y, Y¹, Y², Z, Z¹, and Z² are members independently selected from:
—N— and —CH—;
X³ and Y³ are members independently selected from:
—NH—, —CH₂—, —S—, and —O—;
R, R¹, and R² are members independently selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl;
n, m, p and q are integers independently selected from 0 to 4; and
w is an integer from 0 to 2.

3. A compound of claim 2, wherein said substituted or unsubstituted heteroaryl is selected from substituted or unsubstituted pyridyl, substituted or unsubstituted pyrrolyl, substituted or unsubstituted imidizolyl, substituted or unsubstituted oxazolyl, substituted or unsubstituted thiazolyl, substituted or unsubstituted indolyl, substituted or unsubstituted isoquinolyl, and substituted or unsubstituted purinyl.

4. A compound of claim 2, wherein said substituted or unsubstituted aryl is selected from substituted or unsubstituted phenyl, substituted or unsubstituted naphthyl, and substituted or unsubstituted biphenylmethyl.

5. A compound of claim 2, wherein said substituted or unsubstituted heterocycloalkyl is selected from substituted or unsubstituted pyrrolidinyl, substituted or unsubstituted morpholino, substituted or unsubstituted piperidinyl, and substituted or unsubstituted tetrahydropyranyl.

6. A compound of claim 2, wherein said substituted or unsubstituted cycloalkyl is selected from substituted or unsubstituted cyclopentyl, and substituted or unsubstituted cyclohexyl.

7. A compound of claim 2, wherein said substituted or unsubstituted alkyl is selected from substituted or unsubstituted methyl, substituted or unsubstituted ethyl, substituted or unsubstituted propyl, substituted or unsubstituted butyl, and substituted or unsubstituted pentyl.

8. A compound of claim 2, wherein R—(CH₂)ₙ, R¹—(CH₂)ₘ and R²—(CH₂)ₚ are members independently selected from the moieties represented in FIG. 3.

9. A method of inhibiting or disrupting the interaction between an alpha helix of a first protein and a alpha helix binding pocket of a second protein, said method comprising contacting said second protein with a compound selected from:
A-L-B-L¹-A¹
and 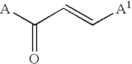
in which
A is a member selected from: 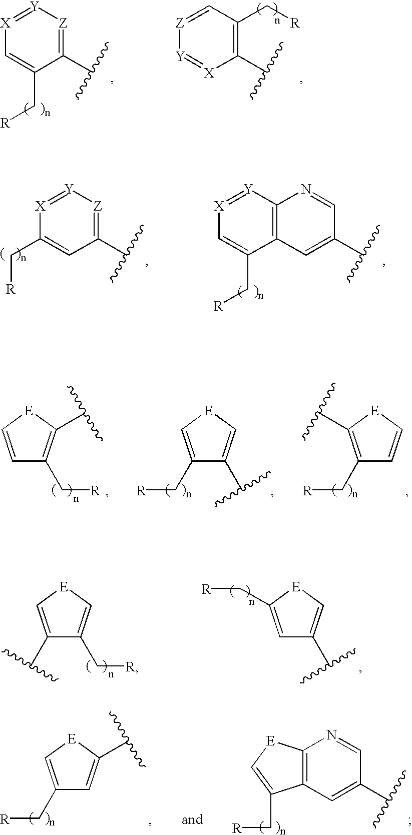
B is a member selected from: 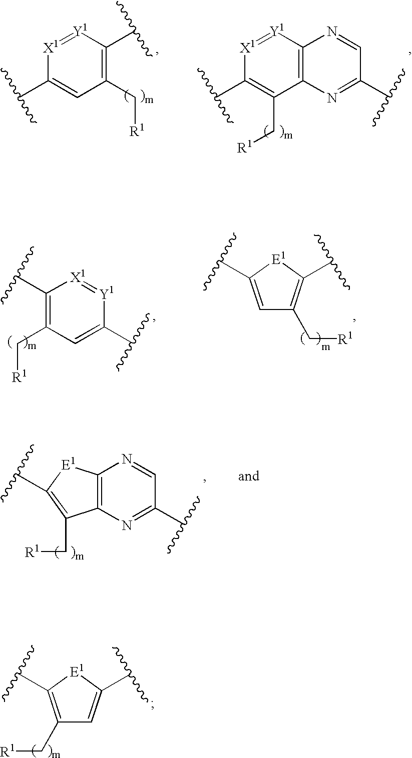
A¹ is a member selected from: 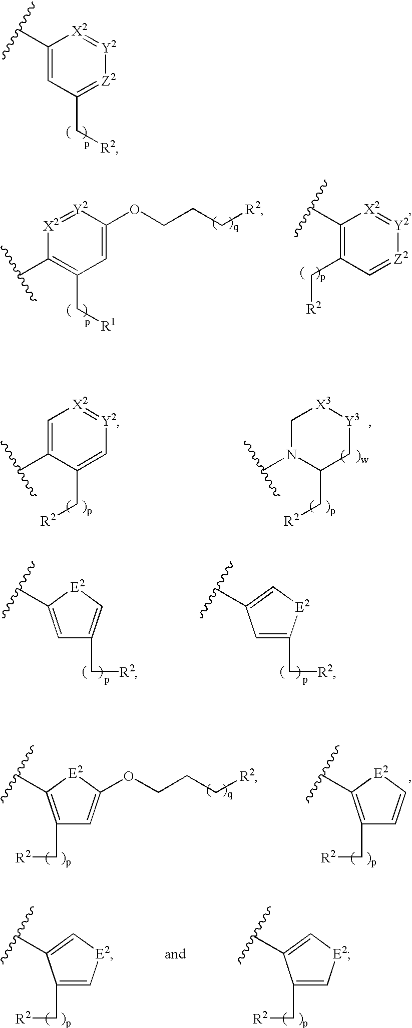
R, R¹, and R² are members independently selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl;
L and L¹ are members independently selected from:
—N═N—, —CH₂—CH₂—, —C═C—, —CH₂—CH₂—, —CH₂—S—, —CH₂—NH—, —NH—CH₂, 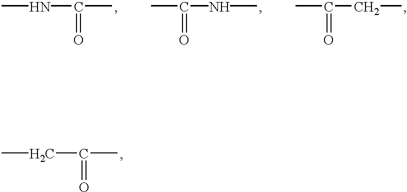
and a single bond;
X, X¹, X², Y, Y¹, Y², Z, Z¹, and Z² are members independently selected from:
—N— and —CH—;
X³, Y³, E, E¹, and E²are members independently selected from:
—NH—, —CH₂—, —S—, and —O—;
E, E¹, and E² members independently selected from:
—NH—, —CH₂—, —S—, and —O—;
n, m, p and q are integers independently selected from 0 to 4; and
w is an integer from 0 to 2.

10. A method of claim 9, wherein said compound has a formula which is a member selected from: 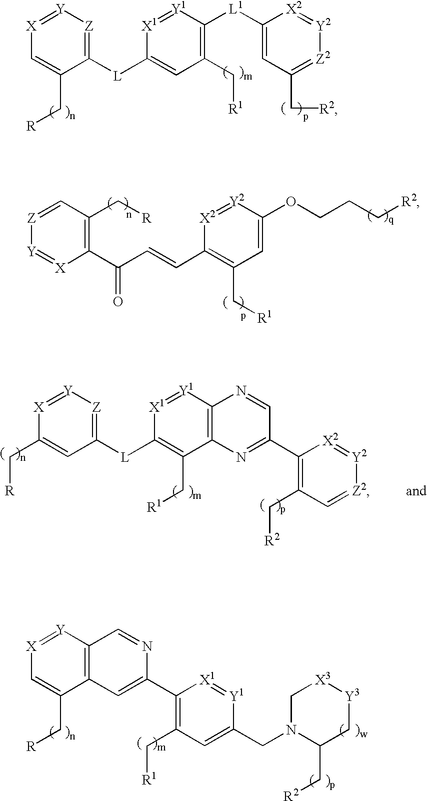
in which
L and L¹ are members independently selected from:
—N═N—, —CH₂—CH₂—, —C═C—, —CH₂—CH₂—, —CH₂—S—, —CH₂—NH—,
—NH—CH₂—, 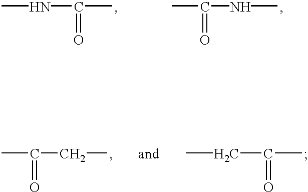
X, X¹, X², Y, Y¹, Y², Z, Z¹, and Z² are members independently selected from:
—N— and —CH—;
X³ and Y³ are members independently selected from:
—NH—, —CH₂—, —S—, and —O—;
R, R¹, and R² are members independently selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl;
n, m, p and q are integers independently selected from 0 to 4; and
w is an integer from 0 to 2.

11. A method of claim 10, wherein said substituted or unsubstituted heteroaryl is selected from substituted or unsubstituted pyridyl, substituted or unsubstituted pyrrolyl, substituted or unsubstituted imidizolyl, substituted or unsubstituted oxazolyl, substituted or unsubstituted thiazolyl, substituted or unsubstituted indolyl, substituted or unsubstituted isoquinolyl, and substituted or unsubstituted purinyl.

12. A method of claim 10, wherein said substituted or unsubstituted aryl is selected from substituted or unsubstituted phenyl, substituted or unsubstituted naphthyl, and substituted or unsubstituted biphenylmethyl.

13. A method of claim 10, wherein said substituted or unsubstituted heterocycloalkyl is selected from substituted or unsubstituted pyrrolidinyl, substituted or unsubstituted morpholino, substituted or unsubstituted piperidinyl, and substituted or unsubstituted tetrahydropyranyl.

14. A method of claim 10, wherein said substituted or unsubstituted cycloalkyl is selected from substituted or unsubstituted cyclopentyl, and substituted or unsubstituted cyclohexyl.

15. A method of claim 10, wherein said substituted or unsubstituted alkyl is selected from substituted or unsubstituted methyl, substituted or unsubstituted ethyl, substituted or unsubstituted propyl, substituted or unsubstituted butyl, and substituted or unsubstituted pentyl.

16. A method of claim 10, wherein R—(CH₂)ₙ, R¹—(CH₂)ₘ and R²—(CH₂)ₚ are members independently selected from the moieties represented in FIG. 3.

17. A pharmaceutical composition comprising a pharmaceutically acceptable excepient in combination with a compound selected from:
A-L-B-L¹-A¹
and 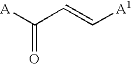
in which
A is a member selected from: 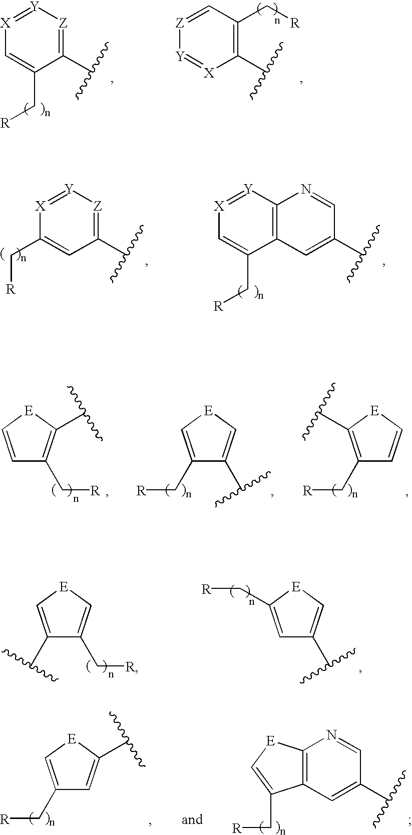
B is a member selected from: 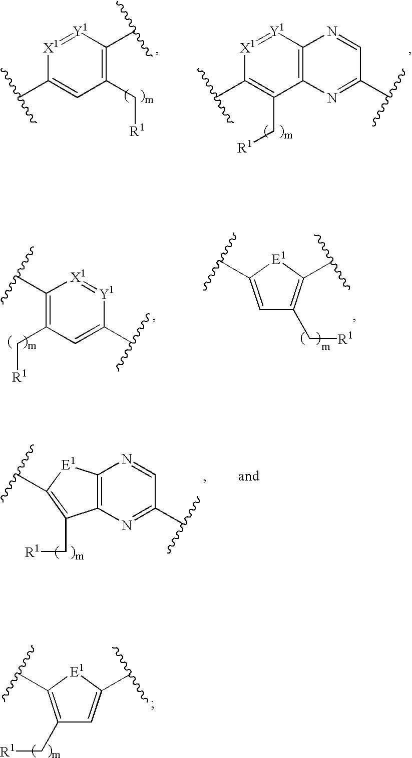
A¹ is a member selected from: 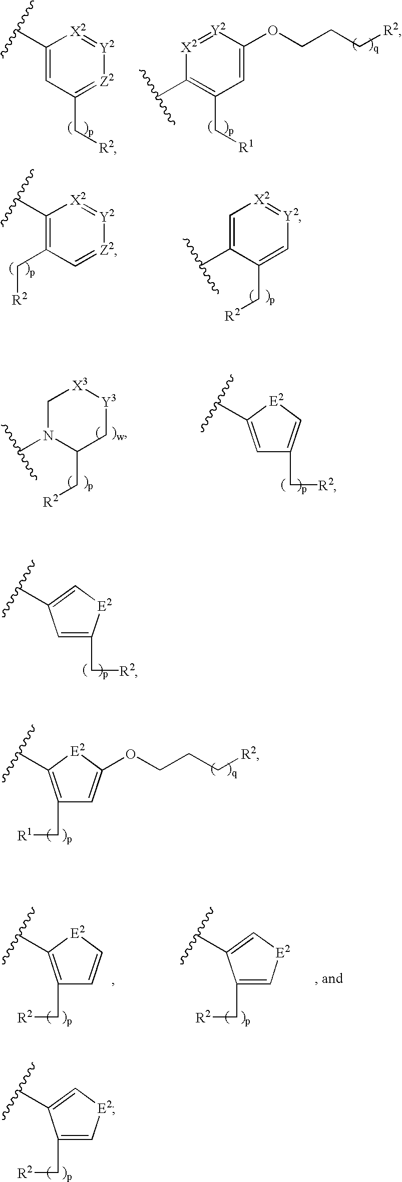
R, R¹, and R² are members independently selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl;
L and L¹ are members independently selected from:
—N═N—, —CH₂—CH₂—, —C═C—, —CH₂—CH₂—, —CH₂—S—, —CH₂—NH—, —NH—CH₂, 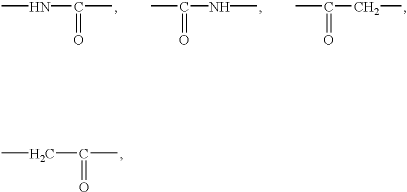
and a single bond;
X, X¹, X², Y, Y¹, Y², Z, Z¹, and Z² are members independently selected from:
—N— and —CH—;
X³, Y³, E, E¹, and E²are members independently selected from:
—NH—, —CH₂—, —S—, and —O—;
E, E¹, and E² members independently selected from:
—NH—, —CH₂—, —S—, and —O—;
n, m, p and q are integers independently selected from 0 to 4; and
w is an integer from 0 to 2.

18. A pharmaceutical composition of claim 17, wherein said compound has a formula which is a member selected from: 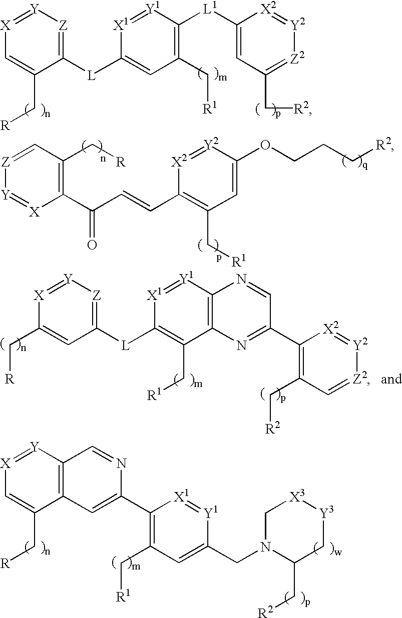
in which
L and L¹ are members independently selected from:
—N═N—, —CH₂—CH₂—, —C═C—, —CH₂—CH₂—, —CH₂—S—, —CH₂—NH—,
—NH—CH₂—, 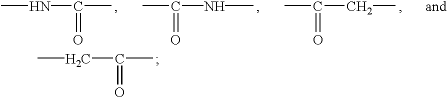
X, X¹, X², Y, Y¹, Y², Z, Z¹, and Z² are members independently selected from:
—N— and —CH—;
X³ and Y³ are members independently selected from:
—NH—, —CH₂—, —S—, and —O—;
R, R¹, and R² are members independently selected from hydrogen, substituted or unsubstituted alkyl, substituted or unsubstituted heteroalkyl, substituted or unsubstituted aryl, substituted or unsubstituted heteroaryl, and substituted or unsubstituted heterocycloalkyl;
n, m, p and q are integers independently selected from 0 to 4; and
w is an integer from 0 to 2.

19. A pharmaceutical composition of claim 18, wherein said substituted or unsubstituted heteroaryl is selected from substituted or unsubstituted pyridyl, substituted or unsubstituted pyrrolyl, substituted or unsubstituted imidizolyl, substituted or unsubstituted oxazolyl, substituted or unsubstituted thiazolyl, substituted or unsubstituted indolyl, substituted or unsubstituted isoquinolyl, and substituted or unsubstituted purinyl.

20. A pharmaceutical composition of claim 18, wherein said substituted or unsubstituted aryl is selected from substituted or unsubstituted phenyl, substituted or unsubstituted naphthyl, and substituted or unsubstituted biphenylmethyl.

21. A pharmaceutical composition of claim 18, wherein said substituted or unsubstituted heterocycloalkyl is selected from substituted or unsubstituted pyrrolidinyl, substituted or unsubstituted morpholine, substituted or unsubstituted piperidinyl, and substituted or unsubstituted tetrahydropyranyl.

22. A pharmaceutical composition of claim 18, wherein said substituted or unsubstituted cycloalkyl is selected from substituted or unsubstituted cyclopentyl, and substituted or unsubstituted cyclohexyl.

23. A pharmaceutical composition of claim 18, wherein said substituted or unsubstituted alkyl is selected from substituted or unsubstituted methyl, substituted or unsubstituted ethyl, substituted or unsubstituted propyl, substituted or unsubstituted butyl, and substituted or unsubstituted pentyl.

24. A pharmaceutical composition of claim 18, wherein R—(CH₂)ₙ, R¹—(CH₂)ₘ and R²—(CH₂)ₚ are members independently selected from the moieties represented in FIG. 3.
